(19) United States
(12) Patent Application Publication
(10) Pub. No.: US 2023/0310207 A1
(43) Pub. Date: Oct. 5, 2023

(54) THERAPY SYSTEM

(71) Applicant: Jeffrey D. Ott, Sylvania, OH (US)

(72) Inventor: Jeffrey D. Ott, Sylvania, OH (US)

(21) Appl. No.: 18/042,125

(22) PCT Filed: Aug. 25, 2021
(86) PCT No.: PCT/US2021/047479

§ 371 Date: Feb. 17, 2023

Related U.S. Application Data
(60) Provisional application No. 63/070,320, filed on Aug. 26, 2020.

(51) Int. Cl. A61F 7/10; A61F 7/02

(52) U.S. Cl. CPC A61F 7/02; A61F 7/10; A61F 2007/0009; A61F 2007/0219; A61F 2007/023; A61F 2007/108

(57) ABSTRACT

A non-invasive, non-surgical, medication free therapy system for application to a targeted body location is provided. The therapy system includes a pouch having a first side and a second side. The first side is configured for contact with the targeted body location. Each of the first and second sides has an inner surface. The inner surfaces of the first and second sides define an internal cavity. A cold temperature source is contained within the internal cavity and is in direct contact with the inner surfaces of the first and second sides. One or more attachment straps are configured for attachment to the second side of the pouch and further configured to position the therapy system adjacent to the targeted body location. The one or more attachment straps still further configured for repeatable and removable attachment to the pouch.

### RELATED APPLICATIONS

This application claims the benefit of U.S. Provisional Pat. Application No. 63/070,320 filed Aug. 26, 2020, the disclosure of which is incorporated herein by reference in its entirety.

### BACKGROUND

Conventional approaches to treating body pains have long included the use of prescription medications, over-the-counter medications, surgery, hot and cold therapy, chiropractic manipulation, physical therapy, or a combination of some or all of these methods. Surgery can be successful at reducing or eliminating body pains, but surgery is invasive, expensive, and can lead to complications and other adverse side effects.

Medications are commonly prescribed to manage pain symptoms, but in most cases, they do not treat any underlying cause of the pain. Medications can also cause allergic reactions and other adverse side effects, and in the case of some painkillers, the medications themselves can be habit forming and addictive. Furthermore, persons who have suffered from dependency in the past cannot use many of the most commonly prescribed medications.

Many current therapeutic solutions consist of applying heat or cold over a broad area, taking the generic approach that heating or cooling the broad area offers some benefit to the actual area incurring pain. Furthermore, current broadly applied cold therapy products often don’t get cold enough or retain their cold temperatures long enough to provide meaningful reduction in inflammation and corresponding pain. And adding to the challenge is the lack of proper devices and/or structures configured to secure the cold therapy products to the affected area of the body.

What is needed is a non-invasive, non-surgical, medication free, therapeutic solution that applies cold therapy targeted directly at pain sources such as inflamed, displaced, or herniated discs, in and around the spinal column and/or swollen muscles and tissue. Additionally, the cold therapy needs to reach lower temperatures and retain these temperatures long enough to supply meaningful reduction in inflammation, thereby directly treating the pain origination cause, and reducing pain.

It would be a significant benefit if cold therapy solutions were comfortable and easily secured in place on a patient at the origins of the incurred pain.

### SUMMARY

It should be appreciated that this Summary is provided to introduce a selection of concepts in a simplified form, the concepts being further described below in the Detailed Description. This Summary is not intended to identify key features or essential features of this disclosure, nor it is intended to limit the scope of the therapy system.

The above objects as well as other objects not specifically enumerated are achieved by a non-invasive, non-surgical, medication free therapy system for application to a targeted body location. The therapy system includes a pouch having a first side and a second side. The first side is configured for contact with the targeted body location. Each of the first and second sides has an inner surface. The inner surfaces of the first and second sides define an internal cavity. A cold temperature source is contained within the internal cavity and is in direct contact with the inner surfaces of the first and second sides. One or more attachment straps are configured for attachment to the second side of the pouch and further configured to position the therapy system adjacent to the targeted body location. The one or more attachment straps still further configured for repeatable and removable attachment to the pouch.

The above objects as well as other objects not specifically enumerated are also achieved by a method of using a non-invasive, non-surgical, medication free therapy system. The method includes the steps of forming a pouch with a first side and a second side, the first side configured for contact with the targeted body location, each of the first and second sides having an inner surface, the inner surfaces of the first and second sides defining an internal cavity, positioning a cold temperature source within the internal cavity and in direct contact with the inner surfaces of the first and second sides, attaching one or more attachment straps to the second side of the pouch, positioning the therapy system adjacent to the targeted body location and wrapping the targeted body location with the one or more attachment straps in a manner such as to secure the therapy system to the targeted body location, wherein the one or more attachment straps are configured for repeatable and removable attachment to the pouch.

Various objects and advantages of the therapy system will become apparent to those skilled in the art from the following Detailed Description, when read in light of the accompanying drawings.

### BRIEF DESCRIPTION OF THE DRAWINGS

FIG. 1 is a perspective view of a therapy system in accordance with the invention.

FIG. 2 is a perspective view of a first side of a two-sided pouch of the therapy system of FIG. 1.

FIG. 3 is a perspective view of a second side of a two-sided pouch of the therapy system of FIG. 1.

FIG. 4 is a cross-sectional view of the two-sided pouch of the therapy system of FIG. 1.

FIG. 5A is a plan view of a first embodiment of an attachment strap of the therapy system of FIG. 1.

FIG. 5B is a side view of the attachment strap of FIG. 5A.

FIG. 6 is perspective view of a first application of the therapy system of FIG. 1.

FIG. 7A is a plan view of a second embodiment of an attachment strap of the therapy system of FIG. 1.

FIG. 7B is a side view of the attachment strap of FIG. 7A.

FIG. 8 is perspective view of a second application of the therapy system of FIG. 1.

FIG. 9 is a plan view of a third embodiment of an attachment strap of the therapy system of FIG. 1.

FIG. 10 is a front perspective view of another application of the therapy system of FIG. 1.

FIG. 11 is a rear perspective view the application of the therapy system of FIG. 10.

### DETAILED DESCRIPTION

The therapy system will now be described with occasional reference to specific embodiments. The therapy system may, however, be embodied in different forms and should not be construed as limited to the embodiments set forth herein. Rather, these embodiments are provided so that this disclosure will be thorough and complete, and will fully convey the scope of the therapy system to those skilled in the art.

Unless otherwise defined, all technical and scientific terms used herein have the same meaning as commonly understood by one of ordinary skill in the art to which the therapy system belongs. The terminology used in the description of the therapy system herein is for describing particular embodiments only and is not intended to be limiting of the therapy system. As used in the description of the therapy system and the appended claims, the singular forms “a,” “an,” and “the” are intended to include the plural forms as well, unless the context clearly indicates otherwise.

Unless otherwise indicated, all numbers expressing quantities of dimensions such as length, width, height, and so forth as used in the specification and claims are to be understood as being modified in all instances by the term “about.” Accordingly, unless otherwise indicated, the numerical properties set forth in the specification and claims are approximations that may vary depending on the desired properties sought to be obtained in embodiments of the therapy system. Notwithstanding that the numerical ranges and parameters setting forth the broad scope of the therapy system are approximations, the numerical values set forth in the specific examples are reported as precisely as possible. Any numerical values, however, inherently contain certain errors necessarily resulting from error found in their respective measurements.

The description and figures disclose a therapy system. The therapy system is a non-invasive, non-surgical, medication free application of cold temperature therapy specifically targeted at pain sources. Known benefits of the application of cold temperature therapies include the reduction and shrinking of inflamed tissues, thereby quickly and effectively reducing pain caused by inflammation of the tissues, stopping and preventing muscle spasms and the associated pain of muscle spasms, numbing the affected tissues causing pain and substantially reducing inflammation and swelling when the cold temperature therapy is implemented. The therapy system includes a flexible, two-sided pouch containing a cold temperature source. The two-sided pouch includes one side configured for direct application to the skin of the affected area and an opposing side manufactured from a continuous loop fabric material that serves to provide an infinite number of attachment points for one or more attachment straps. The one or more attachment straps are reusable and repositionable as needed. The two-sided pouch has a hexagonal cross-sectional shape configured to assist in concentrating the cold originating from the cold temperature source to the desired therapeutic location of the body.

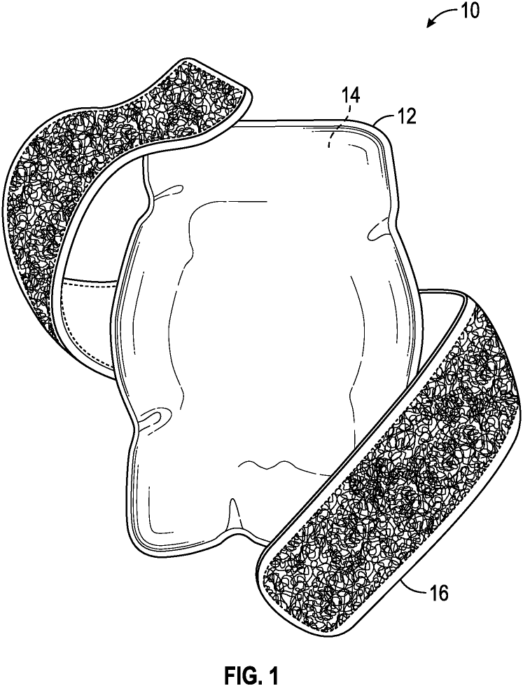

Referring now to the drawings, there is illustrated in FIG. 1 a therapy system generally at 10. The therapy system 10 includes a two-sided pouch 12, a cold temperature source 14 contained within the two-sided pouch 12 and one or more attachment straps 16. The therapy system 10 is repositionable and is configured for attachment to a targeted location of a person’s body, such as the non-limiting examples of spinal regions in the neck, back, and lower lumbar, as well as knees, ankles, elbows, shoulder, back and the like. The term “targeted”, as used herein, is defined to mean a focused bodily area intended for treatment. The therapy system 10 is further configured to position, and secure in place, the cold temperature source 14 directly on the targeted body location in a manner such that the cold temperature source 14 thermally influences the targeted body location.

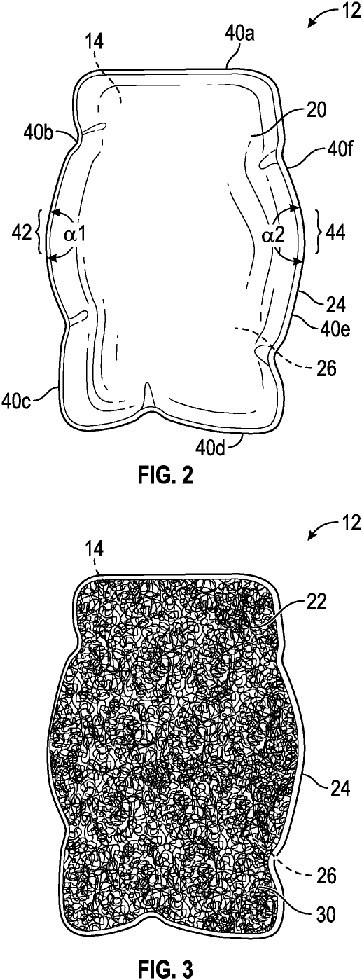

Referring now to FIGS. 2-4, the two-sided pouch 12 is illustrated. The two-sided pouch 12 includes a first side 20 and a second side 22. The two sides 20, 22 are attached together at their perimeters to form a circumferential seam 24. The two sides 20, 22 and the circumferential seam 24 cooperate to define an internal cavity 26. The internal cavity 26 is configured to enclose the cold temperature source 14.

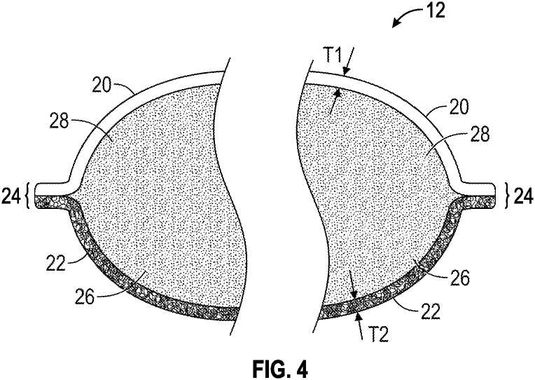

Referring now to FIG. 4, the first side 20 of the two-sided pouch 12 is configured for several functions. First, the first side 20 is configured to remain soft and flexible even though the cold temperature source 14 may be extremely cold or frozen. Second, the first side 20 is configured for contact with human skin. The first side 20 is further configured to convey cold temperature therapy from the cold temperature source 14 to the targeted body location. Finally, the first side 20 of the two-sided pouch 12 is configured to be watertight, that is, to retain the cold temperature source 14 within the two-sided pouch 12. In the illustrated embodiment, the first side 20 is formed as a non-woven fabric based on a polymeric film material. One non-limiting example of a suitable non-woven, polymeric film material is matte olefin alloy, having part number PE2035-1880G-220, manufactured and marketed by Wiman Corporation, headquartered in Sauk Rapids, Minnesota. However, it should be appreciated that other non-woven, polymeric film materials can be used, sufficient for the functions described herein.

Referring again to FIG. 4, the first side 20 has a thickness T1 in a range of from about 0.003 inches to about 0.010 inches. In other embodiments, the thickness T1 can be less than about 0.003 inches or more than about 0.010 inches sufficient for the functions described herein.

Referring again to FIG. 4, the second side 22 of the two-sided pouch 12 is also configured for several functions. First, the second side 22 is configured to remain flexible even though the cold temperature source 14 may be extremely cold or frozen. Second, the second side 22 is configured for removable attachment to the one or more attachment straps 16. The second side 22 of the two-sided pouch 12 is configured to be watertight, that is, to retain the cold temperature source 14 within the two-sided pouch 12. The second side 22 includes a laminated outer surface 30 configured with a plurality of dense loop structures, thereby forming a first side of a hook and loop fastening system. In the illustrated embodiment, the second side 22 is formed as a non-woven fabric based on a polymeric film material. One non-limiting example of a suitable non-woven, polymeric film material is matte olefin alloy, having part number PE2035-1880G-220, manufactured and marketed by Wiman Corporation, headquartered in Sauk Rapids, Minnesota. However, it should be appreciated that other non-woven, polymeric film materials can be used, sufficient for the functions described herein.

Referring again to FIG. 4, the second side 22 has a thickness T2 in a range of from about 0.003 inches to about 0.010 inches. In other embodiments, the thickness T2 can be less than about 0.003 inches or more than about 0.010 inches sufficient for the functions described herein.

Referring again to the embodiment illustrated in FIG. 4, the plurality of dense loop structures formed on the outer surface 30 of the second side 22 extend across the entire outer surface 30 of the second side 22. However, in other embodiments, the plurality of dense loop structures can be discontinuous and/or limited to designated areas of the outer surface 30 of the second side 22, sufficient for removable attachment to the one or more attachment straps 16.

Referring again to FIG. 4, the internal cavity 26 of the two-sided pouch 12 contains the cold temperature source 14. The cold temperature source 14 is configured for several functions. First, the cold temperature source 14 is configured to be reusable and is formed from a material sufficient for many freezing and thawing cycles. Second, the cold temperature source 14 is configured to deliver cold temperature therapy through the first side 20 of the two-sided pouch 12 to the targeted body location in a manner such that the cold temperature therapy thermally influences the targeted body location. In the illustrated embodiment, the cold temperature source 14 is a thermally reactive, gel-based fluid, such as the non-limiting example of a combination of propylene glycol or equivalent, water and a thickening agent. The combination of propylene glycol or equivalent, water and thickening agent advantageously allows the cold temperature source 14 to achieve temperatures approaching zero degrees Fahrenheit while still maintaining flexibility. In addition, it has been found that the combination of the propylene glycol or equivalent, water and thickening agent retains the desired cold temperatures longer than conventional water-based systems or conventional water-based mixtures. In the illustrated embodiment, the thickening agent has the form of a polyacrylate material. In alternate embodiments, the thickening agent can have other suitable forms.

Referring again to the embodiment illustrated in FIG. 4, the cold temperature source 14 is formulated by 1.000 parts propylene glycol, 4.000 parts water and 0.015 parts of a thickening agent such as the non-limiting example of a polyacrylate material. However, it is contemplated that in other embodiments, the cold temperature source 14 can have different concentrations of propylene glycol, water and a thickening agent or that the cold temperature source can be formulated with other suitable components.

Referring again to FIG. 4, the first and second sides 20, 22 of the two-sided pouch 12 are advantageously configured for direct contact with the cold temperature source 14, thereby eliminating the need for a separate cavity liner/layer or other vessel for containing the cold temperature source 14.

Referring again to FIG. 4, in certain instances, the desired cold temperatures of the cold temperature source 14 can be achieved by placing the two-sided pouch 12 in a freezer until the cold temperature source 14 reaches freezer temperature (generally known to be about 5° F.). However, it should be appreciated that the cold temperature source 14 can be chilled in other desired manners. It should be appreciated that use of the thermally reactive, gel-based fluid advantageously allows the cold temperature source 14 to occupy all available portions of the internal cavity 26 within the two-sided pouch 12. However, it should be appreciated that other cold temperature sources 14 can be used.

Referring now to FIG. 2, the two-sided pouch 12 is shown. The two-sided pouch 12 has a hexagonal cross-sectional shape, defined by sides 40a-40f, with the internal cavity 26 formed therein. The intersection of side 40b and side 40c forms a first wing segment 42. In a similar manner, the intersection of sides 40e and 40f forms a second wing segment 44. As will be described in more detail below, the wing segments 42, 44 extend a distance from the two-sided pouch 12 and are configured to assist in the placement of cold temperature source 14 to targeted body areas. More specifically and without being held to the theory, it is believed the wing segments 42, 44 formed by the hexagonal cross-sectional shape of the two-sided pouch 12 better concentrates the cold therapy stemming from the cold temperature source 14 to the desired area of the body. However, it should be appreciated that in other embodiments, the two-sided pouch 12 can have other desired cross-sectional shapes, sufficient for the functions described herein.

Referring again to the embodiment shown in FIG. 2, the sides 40b, 40c of the wing segment 42 form a first angle α1 and the sides 40e, 40f of the wing segment 44 form a second angle α2. In the illustrated embodiment, the first and second angles α1, α2 are each in a range of between 130° and 170°. In alternate embodiments, the first and second angles α1, α2 each can be less than about 130° or more than about 170°, sufficient to assist in the placement of cold temperature source 14 to targeted body areas.

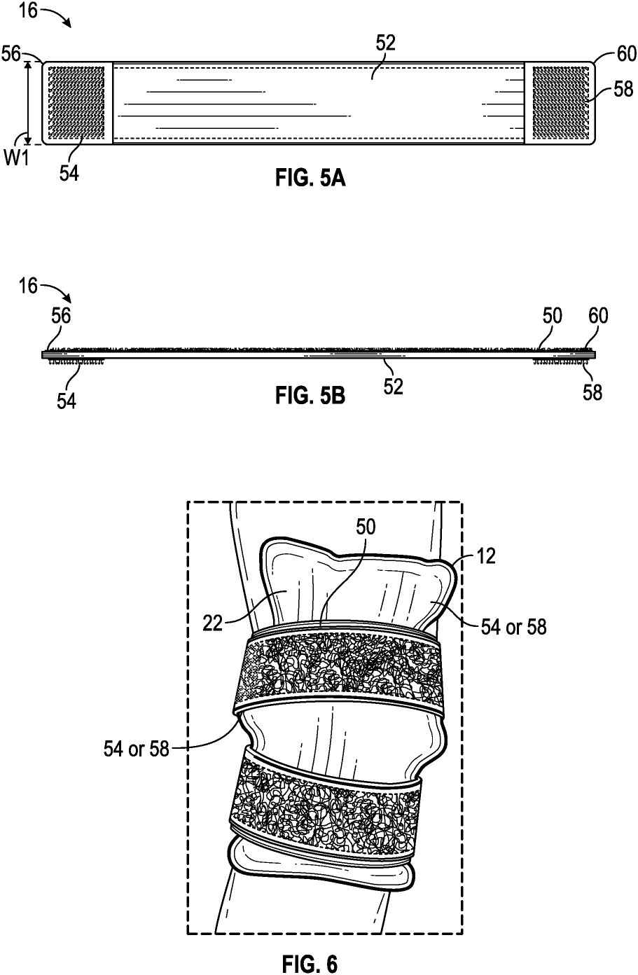

Referring now to FIGS. 5A and 5B, a first embodiment of the attachment strap 16 is illustrated. The attachment strap 16 is configured for several functions. First, the attachment strap 16 is configured for repeatable and removable attachment to the two-sided pouch 12. Second, the attachment strap 16 is configured to position and secure the two-sided pouch 12 to a targeted body location in a manner such that the cold temperature source 14 thermally influences the targeted body location. The attachment strap 16 is formed from a woven, webbed polymeric material and includes a first side 50 and a second side 52. The first side 50 includes a plurality of dense loop structures. The second side 52 includes a first tab 54 positioned at a first end 56 of the attachment strap 16 and a second tab 58 positioned at a second end 60 of the attachment strap 16. Each of the first and second tabs 54, 58 include a plurality of hook structures. It should be appreciated that the attachment strap 16 can have any desired length and that the length can correspond to accommodate the targeted body location.

Referring now to FIG. 6, in operation, the plurality of hook structures positioned on either of the first tab 54 or the second tab 58 is secured to the plurality of dense loop structures positioned on the second side 22 of the two-sided pouch 12. One or more attachment straps 16 are wrapped around the targeted body location (in the illustrated embodiment the targeted body location is a knee) and the plurality of hook structures positioned on the opposing tab 54 is secured to the plurality of dense loop structures positioned on the first side 50 of the attachment strap 16.

Referring again to the embodiment illustrated in FIGS. 5A and 5B, the plurality of dense loop structures extends in a continuous manner across the first side 50 of the attachment strap 16. However, in alternate embodiments, the plurality of dense loop structures can have a discontinuous arrangement and/or can be limited to designated areas of the first side 50, sufficient for repeatable and removable attachment to the two-sided pouch 12 and further sufficient to position and secure the two-sided pouch 12 to a targeted body location in a manner such that the cold temperature source 14 thermally influences the targeted body location. As can be appreciated with conventional hook and loop structures, advantageously, the two-sided pouch 12 and the one or more attachment straps 16 can be used repeatedly and in any relative position.

Referring again to FIG. 5A, the attachment strap 16 has a width w. The width w is configured to provide structural support to the two-sided pouch 12 in an installed arrangement. In the illustrated embodiment, the width w is in a range of from about 0.75 inches to about 3.00 inches. However, in other embodiments, the width w can be less than about 0.75 inches or more than about 3.00 inches, sufficient to provide structural support to the two-sided pouch 12 in an installed arrangement.

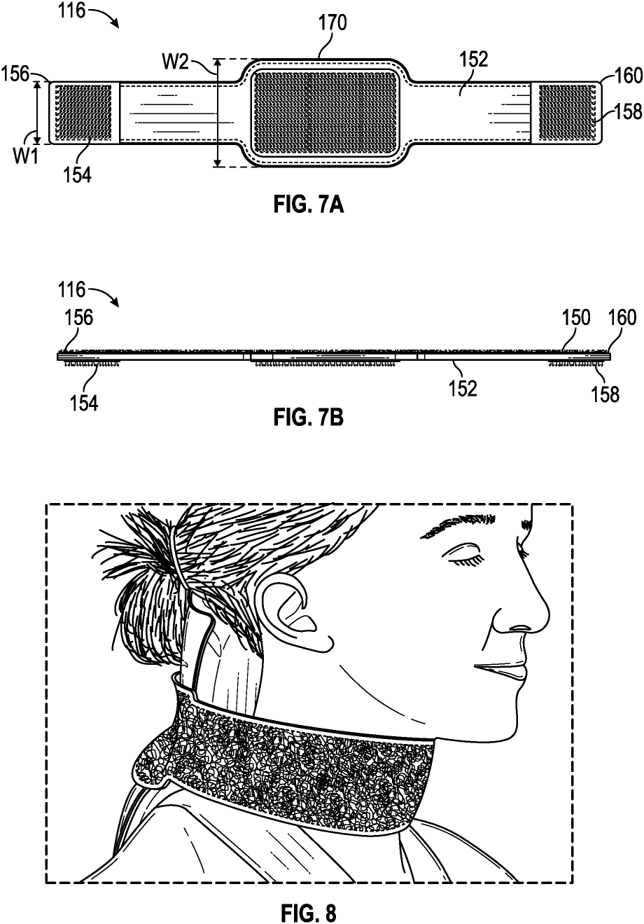

Referring now to FIGS. 7A and 7B, another embodiment of an attachment strap is illustrated at 116. The attachment strap 116 is configured for several functions. First, the attachment strap 116 is configured for repeatable and removable attachment to the two-sided pouch 12. Second, the attachment strap 116 is configured to position and secure the two-sided pouch 12 to a targeted body location in a manner such that the cold temperature source 14 thermally influences the targeted body location. The attachment strap 116 includes a first side 150 and a second side 152. The first side 150 is formed from a polymeric-based material, such as the non-limiting example of neoprene. The second side 152 includes an optional first tab 154 positioned at a first end 156 of the attachment strap 116 and a second tab 158 positioned at a second end 160 of the attachment strap 116. The optional first tab 154 can include a plurality of hook structures. The second tab 158 include a plurality of hook structures. The attachment strap 116 further includes an enlarged support section 170 positioned between the first end 156 and the second end 160. As will be explained in more detail below, the enlarged support section 170 is configured to provide more structural support to the two-sided pouch 12 in an installed position, thereby more effectively spreading contact and contour of the two-sided pouch 12 to the targeted body location, than the attachment strap 16 shown in FIGS. 5A and 5B. The enlarged support section 170 includes a plurality of hook structures positioned on the same side of the attachment strap 116 as the hook structures extending from the first and second tabs 154, 158.

Referring now to FIG. 8, in operation, the plurality of hook structures positioned on the enlarged support section 170 is secured to the plurality of dense loop structures positioned on the second side 22 of the two-sided pouch 12. The two-sided pouch 12 is positioned against the targeted body location (in the illustrated embodiment the targeted body location is a neck). The first end 156 of the attachment strap 116 is wrapped around the targeted body location and in an opposing direction the second end is wrapped around the targeted body location and positioned in contact with the attachment strap 116. The plurality of hook structures positioned on the second tab 158 is secured to the polymeric-based material forming the first side 150 of the attachment strap 116. As can be appreciated with conventional hook and loop structures, advantageously, the two-sided pouch 12 and the attachment strap 116 can be used repeatedly.

Referring again to FIG. 7A, the first and second ends 156, 160 of the attachment strap 116 has a width wl and the enlarged support section 170 has a width w2. The width w2 is greater than the width w1. In the illustrated embodiment, the width w1 is in a range of from about 0.75 inches to about 3.00 inches and the width w2 is in a range of from about 1.00 inch to about 4.00 inches. However, in other embodiments, the width w1 can be less than about 0.75 inches or more than about 3.00 inches and the width w2 can be less than about 1.00 inch or more than about 4.00 inches, sufficient for the functions described herein.

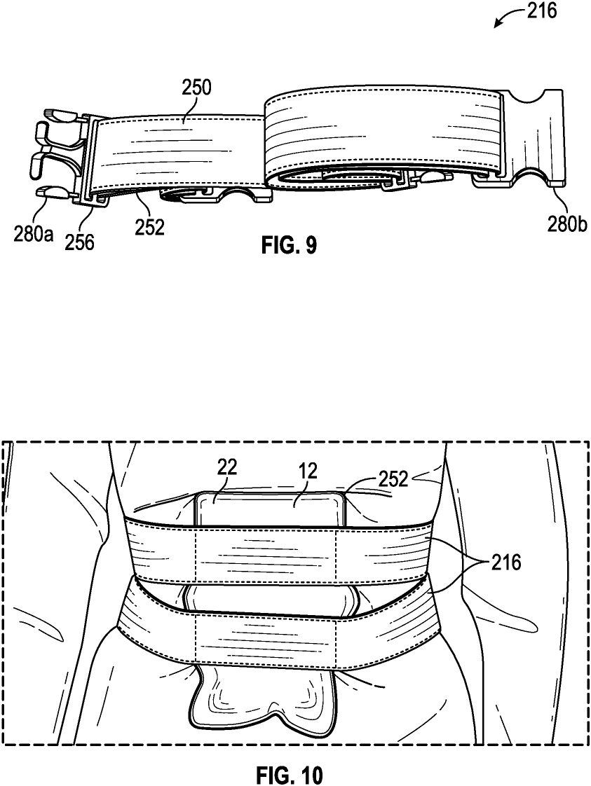

Referring now to FIG. 9, a third embodiment of an attachment strap is illustrated at 216. The attachment strap 216 includes a first side 250, a second side 252 and opposing buckle components 280a, 280b. The second side 252 includes a plurality of hook tabs (not shown for purposes of clarity). The plurality of hook tabs is configured to engage the dense loop structures positioned on the second side 22 of the two-sided pouch 12. The opposing buckle components 280a, 280b are configured for attachment to each other and further configured to allow adjustment to the length of the attachment strap 216. In the illustrated embodiment, the buckle component 280a has the form of a tongue and the buckle component 280b has the form of a receiver, configured to receive the buckle component 280a. However, in other embodiments, any desired buckles can be used sufficient for attachment to each other and further sufficient to allow adjustment to the length of the attachment strap 216.

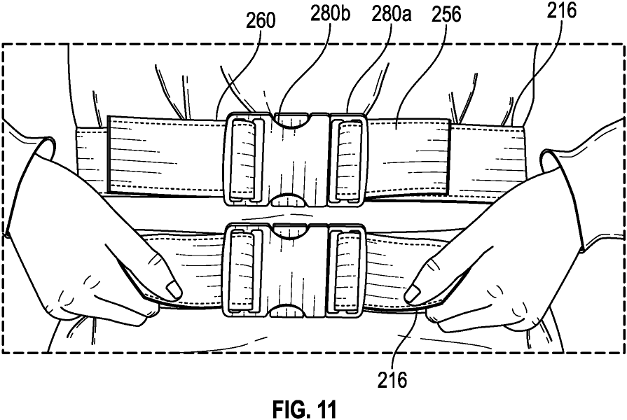

Referring now to FIGS. 10 and 11, in operation, the plurality of hook tabs positioned on the second side 252 is secured to the plurality of dense loop structures positioned on the second side 22 of the two-sided pouch 12. The two-sided pouch 12 is positioned against the targeted body location (in the illustrated embodiment the targeted body location is a portion of a back). A first end 256 of the attachment strap 216 is wrapped around the targeted body location and in an opposing direction the second end 260 is wrapped around the targeted body location. The opposing buckle components 280a, 280b are joined together and the length of the attachment strap 216 is adjusted in a manner such that the cold temperature source 14 adjoins and thermally influences the targeted body location. While the embodiment shown in FIGS. 10 and 11 illustrate the use of two (2) attachment straps 216, it should be appreciated that in other embodiments, more or less than two (2) attachment straps 216 can be used.

The therapy system 10 provides many benefits, although all benefits may not be present in all embodiments. First, the therapy system 10 provides application of a flexible two-sided pouch 12 containing the cold temperature source 14, to a desired and targeted body location. Second, the two-sided pouch 12, having one side equipped with a continuous loop material, provides an infinite number of attachment points with one or more attachment straps 16, 116, 216. Third, the one or more attachment straps 16, 116, 216 are reusable and repositionable as needed. Fourth, the hexagonal cross-sectional shape of the two-sided pouch 12 assists in concentrating the cold stemming from the cold temperature source 14 to the targeted body location. Fifth, when used, the therapy system 10 provides for movement and activity by the user, thereby eliminating the need for the user to be in a stationary orientation.

While the therapy system 10 shown in FIGS. 1-4, 5A, 5B, 6, 7A, 7B and 8-11 is described above in the context of applying cold to targeted body locations, it is contemplated that the therapy system 10 can be configured to apply other forms of therapy to targeted body locations. Non-limiting examples of other forms of therapy include heat, ultrasonic stimulation and the like.

In accordance with the provisions of the patent statutes, the principle and mode of operation of the therapy system have been explained and illustrated in a certain embodiment. However, it must be understood that the therapy system may be practiced otherwise than as specifically explained and illustrated without departing from its spirit or scope.

What is claimed is:

1. A non-invasive, non-surgical, medication free therapy system for application to a targeted body location, the therapy system comprising:
a pouch having a first side and a second side, the first side configured for contact with the targeted body location, each of the first and second sides having an inner surface, the inner surfaces of the first and second sides defining an internal cavity;
a cold temperature source contained within the internal cavity and in direct contact with the inner surfaces of the first and second sides; and
one or more attachment straps configured for attachment to the second side of the pouch and further configured to position the therapy system adjacent to the targeted body location, the one or more attachment straps still further configured for repeatable and removable attachment to the pouch.

2. The therapy system of claim 1, wherein the pouch has a hexagonal cross-sectional shape.

3. The therapy system of claim 1, wherein the first side is formed from a non-woven fabric based on a polymeric film material.

4. The therapy system of claim 3, wherein the polymeric film material is matte olefin alloy.

5. The therapy system of claim 1, wherein the second side is a laminated structure formed from a polymeric film material having a plurality of dense loop structures on an outer surface.

6. The therapy system of claim 5, wherein the polymeric film material is matte olefin alloy.

7. The therapy system of claim 1, wherein the cold temperature source is configured for freezing and thawing cycles.

8. The therapy system of claim 1, wherein the cold temperature source is a thermally reactive, gel-based fluid, and is formed from a combination of propylene glycol or equivalent, water and a thickening agent.

9. The therapy system of claim 1, wherein the one or more attachment strap include opposing ends, each having tabs with a plurality of hook structures.

10. The therapy system of claim 1, wherein the one or more attachment straps include buckles at opposing ends.

11. A method of using a non-invasive, non-surgical, medication free therapy system, the method comprising:
forming a pouch with a first side and a second side, the first side configured for contact with the targeted body location, each of the first and second sides having an inner surface, the inner surfaces of the first and second sides defining an internal cavity;
positioning a cold temperature source within the internal cavity and in direct contact with the inner surfaces of the first and second sides;
attaching one or more attachment straps to the second side of the pouch;
positioning the therapy system adjacent to the targeted body location; and
wrapping the targeted body location with the one or more attachment straps in a manner such as to secure the therapy system to the targeted body location, wherein the one or more attachment straps are configured for repeatable and removable attachment to the pouch.

12. The method of claim 11, including the step of forming the pouch with a hexagonal cross-sectional shape.

13. The method of claim 11, including the step of forming the first side from a non-woven fabric based on a polymeric film material.

14. The method of claim 13, wherein the polymeric film material is matte olefin alloy.

15. The method of claim 11, including the step of forming the second side as a laminated structure based a polymeric film material.

16. The method of claim 15, wherein the polymeric film material is matte olefin alloy laminate.

17. The method of claim 11, including the step of configuring the cold temperature source for freezing and thawing cycles.

18. The method of claim 11, including the step of forming the cold temperature source from a thermally reactive, gel-based fluid that is a combination of propylene glycol or equivalent, water and a thickening agent.

19. The therapy system of claim 11, including the step of positioning tabs at opposing ends, each of the tabs having a plurality of hook structures.

20. The therapy system of claim 11, wherein the one or more attachment straps each include buckles at opposing ends.
